(19) United States
(12) Patent Application Publication
(10) Pub. No.: US 2022/0028565 A1
(43) Pub. Date: Jan. 27, 2022

(54) PATIENT SUBTYPING FROM DISEASE PROGRESSION TRAJECTORIES

(71) Applicant: KONINKLIJKE PHILIPS N.V., EINDHOVEN (NL)

(72) Inventors: Nikhil GALAGALI, Mountain View, MA (US); Minnan XU, Cambridge, MA (US); Bryan CONROY, Garden City South, NY (US); Asif RAHMAN, Cambridge, MA (US); David Paul NOREN, Sharon, MA (US)

(21) Appl. No.: 17/276,708

(22) PCT Filed: Sep. 17, 2018
(86) PCT No.: PCT/EP2019/074878

§ 371 Date: Mar. 16, 2021

Related U.S. Application Data
(60) Provisional application No. 62/732,309, filed on Sep. 17, 2018.

(51) Int. Cl. G16H 70/60; G16H 10/60

(52) U.S. Cl. CPC G16H 70/60; G16H 10/60

(57) ABSTRACT

A method of determining patient subtyping from disease progression trajectories, including: extracting patient data and related time stamps from patient record data related to a disease, wherein the extracted patient data is incomplete and irregular; building a continuous-time disease progression model based upon the extracted patient data; and building a mixture model for clustering of patient disease trajectory subtypes.

### TECHNICAL FIELD

Various exemplary embodiments disclosed herein relate generally to patient subtyping from disease progression trajectories.

### BACKGROUND

Patient subtyping can be used to make improved outcome predictions, understand disease etiologies, plan customized treatments, and design efficient clinical trials.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a method of determining patient subtyping from disease progression trajectories, including: extracting patient data and related time stamps from patient record data related to a disease, wherein the extracted patient data is incomplete and irregular; building a continuous-time disease progression model based upon the extracted patient data; and building a mixture model for clustering of patient disease trajectory subtypes.

Various embodiments are described, further including extracting clinical insights regarding disease progression from the patient disease trajectory subtypes.

Various embodiments are described, further including displaying clustered extracted patient data and a disease state diagram.

Various embodiments are described, further including predicting a patient observation by inputting patient data into the mixture model to determine the patient's disease trajectory.

Various embodiments are described, further including recommending a patient intervention based upon the predicted patient observation.

Various embodiments are described, wherein the continuous-time disease progression model is a continuous Markov chain.

Various embodiments are described, wherein the continuous-time disease progression model parameters are determined based upon training data.

Various embodiments are described, wherein the mixture model is trained using a maximum likelihood approach.

Various embodiments are described, wherein the mixture model is trained using a Bayesian approach.

Further various embodiments relate to a non-transitory machine-readable storage medium encoded with instructions for determining patient subtyping from disease progression trajectories, the non-transitory machine-readable storage medium including: instructions for extracting patient data and related time stamps from patient record data related to a disease, wherein the extracted patient data is incomplete and irregular; instructions for building a continuous-time disease progression model based upon the extracted patient data; and instructions for building a mixture model for clustering of patient disease trajectory subtypes.

Various embodiments are described, further including extracting clinical insights regarding disease progression from the patient disease trajectory subtypes.

Various embodiments are described, further including displaying clustered extracted patient data and a disease state diagram.

Various embodiments are described, further including predicting a patient observation by inputting patient data into the mixture model to determine the patient's disease trajectory.

Various embodiments are described, further including recommending a patient intervention based upon the predicted patient observation.

Various embodiments are described, wherein the continuous-time disease progression model is a continuous Markov chain.

Various embodiments are described, wherein the continuous-time disease progression model parameters are determined based upon training data.

Various embodiments are described, wherein the mixture model is trained using a maximum likelihood approach.

Various embodiments are described, wherein the mixture model is trained using a Bayesian approach.

### BRIEF DESCRIPTION OF THE DRAWINGS

In order to better understand various exemplary embodiments, reference is made to the accompanying drawings, wherein:

FIG. 1 illustrates the overall disease progression model;

FIG. 2 illustrates prototypical results from the mixture-model model with three clusters; and

FIG. 3 is flow diagram illustrating the uses of the model by clinicians and hospitals to understand how disease development and management varies across different subtypes of any acute or chronic disease.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

Patient subtyping is an important topic in medical informatics. Subtyping may be used to make improved outcome predictions, understand disease etiologies, plan customized treatments, and design efficient clinical trials. Traditionally, subtyping has been based either on summaries (e.g., mean) of patient's entire set of observations or summaries over blocks of fixed time-intervals. These approaches result in vectors of fixed size for all patients, which are then amenable to well-known clustering approaches such as k-means clustering, hierarchical clustering etc. Native data in electronic medical records (EMR), however, are almost always incomplete and irregularly sampled over varying time intervals. Such data may also be very noisy. The incompleteness of data is typically dealt with by an imputation step to produce a complete data set of constant dimensions. Although practically useful, clustering with a summary-based fixed-dimensional data set ignores rich information in the temporal patterns of clinical observations/interventions. Clustering approaches that work with data in their native form (clinical markers and their time-stamps) can lead to more nuanced disease subtyping that fully exploits the complex information contained in clinical markers along with their temporal patterns. For example, many diseases may have a series of states, and understand which state a patient is in may provide insights to the severity and treatment of the disease.

For example, in intensive care units (ICUs), clinicians may be treating a number of acute patients and may receive large amounts of data regarding that patient's condition. In such a stressful situation the clinician may benefit from automated modelling tools that use the large amounts of data to suggest a course of action to take with the patient. Further, these acute patients may have a complicated set of conditions to be treated and considered. Accordingly, finding disease subtypes for these patients will allow for better real-time and customized treatment based upon real-time data that is streaming in along with patient background data.

Methods that subtype entire disease progression trajectories based on the temporal patterns of clinical markers have been developed in recent years. Existing methods, however, suffer from a problem: they treat the evolution of clinical markers as one homogeneous process. This assumes a single model for the patient's disease evolution, and does not account for the fact that a patient may show different observation dynamics in different time periods. In reality, all diseases have different states in their progression, manifesting in different rates at which the disease progresses during a patient's lifetime. In practice, observations of patients shows that there are different disease subtypes where patients progress through the various disease states at different rates.

For example, adult respiratory distress syndrome (ARDS) may be caused by pneumonia as one subtype, by sepsis as another subtype, etc. So, one or a plurality of comorbidities may be ARDS subtypes.

Embodiments described herein include two elements: a model for incomplete, irregular clinical markers that is built using a continuous-time disease progression model; and a mixture model for soft clustering of patient disease trajectories. Both of these will be described in detail below.

First, the structure of the dataset that is available in medical records and the resulting challenges it presents for subtyping are described. Consider that medical records include data from N patients, each associated with their time course of observations given by Yₙ ≡{Y_{n,t}_{₁}, Y_{n,t}_{₂}, . . . Y_{n,t}_{ₙ}}. Here, Y_{n,t}_{ᵢ} is the vector of observations at time tᵢ and Yₙ is the trajectory of observation vectors of patient n. The length of each Y_{n,t}_{ᵢ} is D, where D is the number of features that could be observed. For example, if the data includes the heart rate, blood pressure, and respiratory rate measurements of patients, D would be three. Usually, only a subset of the D features are actually observed at any time, with the specific features observed being different at each time point. This results in an incomplete observation set with many feature observations missing. As such, patient observations are made when appropriate—when the patient appears for a routine check-up or when clinicians ask for specific tests/measurements. As a result, the trajectories of patient observations do not synchronize in time or the type of features that are observed.

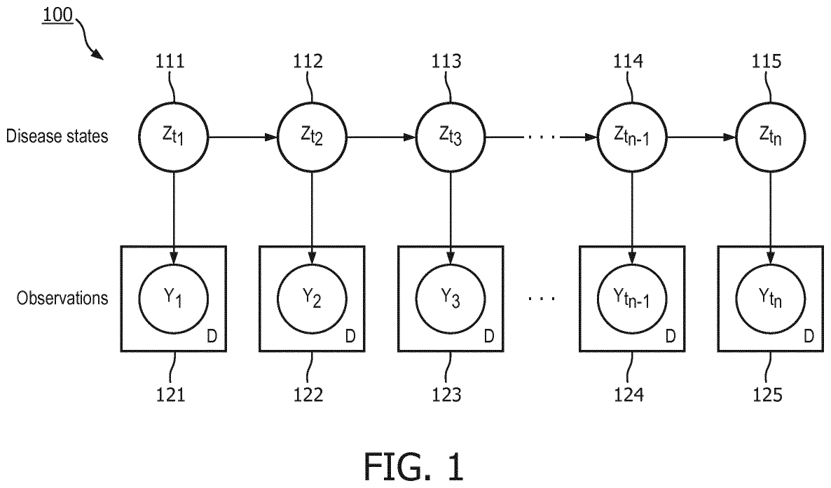

Disease evolution is fundamentally a continuous process: a patient's disease state transitions may happen at any time with the chance of state transition between any two time points higher if the time interval is longer. Thus a continuous-time Markov chain is used to model the evolution of a patient's disease state. The disease state of patient n at time tᵢ is denoted as Z_{n,t}_{ᵢ} and takes one of a set of discrete values. The disease state is naturally hidden, i.e., we never get to observe the actual disease state. In fact, the precise definition of the disease states is apriori unknown. The disease states can be learned from data in an unsupervised manner and subsequently the states interpreted based on the parameters that describe the states. The observation vectors Y_{n,t}_{ᵢ} are surrogates of the underlying disease state Z_{n,t}_{ᵢ}. To reflect this behavior in the model described herein, the observations are modelled by a conditionally independent probability model P(Y_{n,t}_{ᵢ}|Z_{n,t}_{ᵢ}), where observation Y_{n,t}_{ᵢ} is independent of all other observations Y_{n,t}_{ⱼ} given the current disease state Z_{n,t}_{ᵢ}. Overall, the model for the patient's observation trajectory can be described by the continuous-time hidden Markov model (CT-HMM) shown in FIG. 1. A CT-HMM models the temporal evolution of disease states and the state-dependent observation vectors. FIG. 1 illustrates the disease states Zₜ_{₁} to Zₜ_{ₙ} 111-115 and corresponding observations Yₜ_{₁} to Yₜ_{ₙ} 121-125.

As mentioned in the above paragraph, the evolution of disease state P(Z_{n,t}_{ᵢ}|Z_{n,t}_{_{i−1}}) is modeled with a continuous-time Markov chain. A continuous-time Markov chain is a continuous-time process on a state-space (here the different disease states) satisfying the Markov property. This means that if [symbol] _{Z(s)} is all the information about the history of the disease state Z up to time s and s≤t, then Z(t) is independent of all Z(t′), where t′<s, given Z(s). Mathematically, this can be expressed as

P(Z(t)=k| [symbol] _{Z(s)})=P(Z(t)=k|Z(s)). (1)

Further, it is assumed that the process to be time-homogeneous, so that

P(Z(t)=k|Z(s))=P(Z(t−s)=k|Z(0)). (2)

Equations 1 and 2 define a time-homogeneous continuous-time Markov chain and model the disease state evolution in the model. K different disease states are allowed in the model. The transition probability of moving from state a to state b over time Δ in a continuous-time Markov chain is given by

P(Z_{n,t}_{ᵢ}=b|Z_{n,t}_{_{i−1}}=a,tᵢ−tᵢ₋₁=Δ;Q)=expm(ΔQ)_{ab}, (3)

where Q is the generator matrix of the Markov process and expm is the matrix exponential. The probability of the initial state P(Z_{n,t}_{₁}) is parameterized by π={π₁, π₂, . . . , π_{K}} and given by

$${π}_{k} {=}^{△} P ( {Z}_{n , {t}_{1}} = k ) , k = 1 , 2 , … , K \qquad ( 4 )$$

The state-observation trajectory of a patient may be modeled by the continuous-time hidden Markov model shown in FIG. 1. The probability of the trajectory of patient n is given by

P(Z_{n,t}_{₁}_{:t}_{ₙ},Y_{n,t}_{₁}_{:t}_{ₙ})=P(Y_{n,t}_{₁}_{:t}_{ₙ}|Z_{n,t}_{₁}_{:t}_{ₙ})P(Z_{n,t}_{₁}_{:t}_{ₙ}) (5)

Throughout this disclosure, the notation l:r is used to denote all values ranging from l to r (inclusive of both boundaries). Due to the conditional independence property of the CT-HMM in FIG. 1, the joint probability of the states and observations can be written as

$$P ( {Z}_{n , {t}_{1} : {t}_{n}} , {Y}_{n , {t}_{1} : {t}_{n}} ) = {\prod }_{t = {t}_{1}}^{{t}_{n}} P ( {Y}_{n , t} \vert {Z}_{n , t} ) P ( {Z}_{n , {t}_{1} : {t}_{n}} ) \qquad ( 6 )$$

A common modeling choice that is incorporated in the model is that the features are conditionally independent given the corresponding disease state, i.e.,

$$P ( {Y}_{n , t} \vert {Z}_{n , t} ) = {\prod }_{d = 1}^{D} P ( {Y}_{n , t , d} \vert {Z}_{n , t} ) \qquad ( 7 )$$

The choice of the conditional distribution of the observation Y_{n,t,d} given the disease state Z_{n,t} can be made as per the context. If recorded data only indicates whether a certain feature was observed or not, then the individual features Y_{n,t,d} can be modeled by a Bernoulli random variable. An example of this with healthcare data is when ICD9 code assignments are recorded along with their time stamps. In contrast, if the magnitude of feature observations are available, then continuous distributions like the Gaussian or the log-normal distributions could be used. Another approach to work with numerical values is to bin the values and then model the probability of observing values in the bins through a categorical distribution. This is the approach used herein. Specifically, if it is assumed that a feature d can fall into one off bins, the conditional probability of the jth bin is given by

$$P ( {Y}_{n , t , d} \vert {Z}_{n , t} = k ) = {\prod }_{j = 1}^{J} {w}_{k , d , j}^{{1}_{{Y}_{n , t , d} = j}} , \qquad ( 8 )$$

where k refers to the disease state at time t and w_{k,d,1:J} are the parameters of the categorical distribution of the feature d given disease state k. By construction Σⱼ w_{k,d,j}=1. In case of missing feature observation, that observation is marginalized from the model.

The disease states in a patient's observation timeline are unknown. Thus, the patient's observation trajectory may be quantified by marginalizing the disease state out of Equation 6, giving

$$P ( {Y}_{n , {t}_{1} : {t}_{n}} ) = {\sum }_{{Z}_{n , {t}_{1} : {t}_{n}}} P ( {Z}_{n , {t}_{1} : {t}_{n}} , {Y}_{n , {t}_{1} : {t}_{n}} ) . \qquad ( 9 )$$

This is referred to as the likelihood under the patient's disease trajectory model. A disease trajectory model is parameterized by π, Q, and w. Equipped with a likelihood model of the patient's observation trajectory, now a measure of similarity between trajectories of different patients may be described. Patients whose observation trajectories are more probable under a disease trajectory model than other trajectory models can be considered to be similar trajectories. This measure of similarity works even in cases when patients' count of observations, time span of observation window, and the times when different features are observed are different. This measure of similarity works even in cases when patients' observation trajectories do not match in the time stamps of observations and the features observed. In other words, the patient observation trajectory likelihood based similarity metric allows comparison between trajectories when the patient observations are irregular, incomplete and when the observation trajectories across patients are asynchronous.

We subtype patients into different clusters using the mixture model. Consider that we are interested in identifying M subtypes among the patients. In a mixture model, the probability of a patient observation trajectory is the weighted sum of the probability of observing the trajectory in each of the mixture components, where the weights sum to 1. Mathematically, the probability of a trajectory in a mixture model is expressed as

$$P ( {Y}_{n , {t}_{1} : {t}_{n}} ) = {\sum }_{m = 1}^{M} P ( m ) P ( {Y}_{n , {t}_{1} : {t}_{n}} \vert m ) , \qquad ( 10 )$$

where P(Y_{n,t}_{₁}_{:t}_{ₙ}|m) is the probability of observing the trajectory Y_{n,t}_{₁}_{:t}_{ₙ} given that the patient belongs to subtype m and P(m) is the prior probability of subtype m. Thus, the joint distribution of patient n's subtype assignment mₙ and his/her trajectory Y_{n,t}_{₁}_{:t}_{ₙ} is given by

P(mₙ,Y_{n,t}_{₁}_{:t}_{ₙ})=P(mₙ)P(Y_{n,t}_{₁}_{:t}_{ₙ}|mₙ). (11)

Here mₙ∈{1, 2, . . . , M} and P(Y_{n,t}_{ᵢ}_{:t}_{ₙ}|mₙ) are evaluated using the disease trajectory model with parameters (πₘ_{ₙ}, Qₘ_{ₙ}, wₘ_{ₙ}) corresponding to subtype mₙ. To infer the subtypes, patient subtype assignments and subtype parameters are identified so as to maximize the joint probability of the subtype assignment and the conditional observation trajectory probability over all patients. Mathematically, assuming independence of patients, the objective used to identify the subtypes is

{m*_{1:N},π*_{1:M},Q*_{1:M},w*_{1:M}}=

$${arg max}_{{m}_{1 : N} , {π}_{1 : M} , {Q}_{1 : M} , {w}_{1 : M}} {\prod }_{n = 1}^{N} P ( {m}_{n} , {Y}_{n , {t}_{1} : {t}_{n}} ) \qquad ( 12 )$$

With the above objective, each patient gets assigned the subtype with the highest posterior probability. One could instead also use the maximum likelihood of the mixture model (10) as the objective, producing a soft clustering of patients. A natural choice for the prior probabilities of subtype assignment is to assume a uniform distribution (P(mₙ)=1/M) for each mₙ, i.e., each patient is apriori equally likely to belong to any subtype. This translates into patients in the training data being assigned into subtypes and subtype parameters learnt so as to maximize the product of the conditional likelihood (Πₙ₌₁^{N} P(Y_{n,t}_{₁}_{:t}_{ₙ} |mₙ)) of all patient trajectories. Of course, in cases where the modeler would like to relax this assumption, the prior distribution P(mₙ) of the subtype assignment can also be learnt from available data. Once the optimal parameters are learnt from the training data, for a new patient not in the training data, the subtype is identified as the subtype with the highest posterior probability for that patient.

The different steps involved in training the subtyping model are now presented. The subtyping model may be trained by maximizing the objective (Equation 12) using a coordinate ascent optimization algorithm. The algorithm (see Algorithm 1 below) includes two alternating steps: Step 1, when each patient trajectory gets assigned to the subtype with the highest posterior probability, and Step 2, when all patients assigned to a subtype are used to optimize the parameters of that subtype. The precise mathematical forms of the two steps are given in Algorithm 1. In Step 2 of the algorithm 1, parameters of each subtype are learned by training the disease trajectory model described above. The solution of each maximization problem in Step 2 is a maximum likelihood estimate of the subtype parameters with the data assigned to that subtype. As was explained above, the likelihood of a patient trajectory P(Y_{n,t}_{₁}_{:t}_{ₙ}) can be realized by marginalizing the hidden disease states from the joint probability distribution of the observations and the disease states (Equation 9). Thus, the optimization in Step 2 can be solved with the expectation maximization algorithm. The E- and M-steps in optimizing equation 14 are given in Algorithm 2 below.

Algorithm 1 Patient subtyping algorithm

| 1: | Given: Number of subtypes M and observation trajectories | |
|---|---|---|
| | {Yₙ ≡ Y_{n,t}_{₁}_{:t}_{ₙ}} of N patients | |
| 2: | Repeat until convergence: | |
| 3: | Step 1: | ${m}_{1 : n}^{*} = arg {max}_{{m}_{1 : n}} P ( {Y}_{1 : n} , {m}_{1 : n} , \overline{π} , \overline{Q} , \overline{w} ) \qquad ( 13 )$ |
| 4: | Step 2: | For m = 1 to M: |
| | | $\begin{matrix}\begin{matrix}\{ {π}_{m}^{*} , {Q}_{m}^{*} , {w}_{m}^{*} \} = \\ {arg max}_{{π}_{m} , {Q}_{m} , {w}_{m}} {\prod }_{n ∈ N ( m )}^{ } P ( {Y}_{n , {t}_{1} : {t}_{i}} ; {π}_{m} , {Q}_{m} , {w}_{m} ) , \\ \text{where} \text{N} \text{(} \text{m} \text{) are patients assigned to subtype} \text{m}\end{matrix} & ( 14 )\end{matrix}$ |

Algorithm 2 Disease trajectory learning algorithm

| 1: | Given: Trajectories Y ≡ {Y_{n∈N(m)}} of patients assigned to subtype m | | |
|---|---|---|---|
| 2: | Zₙ ≡ Z_{n,t}_{₁}_{:}t_{ₙ} and Z ≡ {Z_{n∈N(m)}} | | |
| 3: | Repeat until convergence: | | |
| 4: | E-Step: | [symbol] p_{(Z,Z(t)\|Y;π′,Q′,w′)} log P (Y, Z, Z(t); π, Q, w) | (15) |
| | | = [symbol] p_{(Z,Z(t)\|Y;π′,Q′,w′)} log P (Z, Z(t); Q) | |
| | | + [symbol] p_{(Z\|Y;π′,Q′,w′)} log P (Y\|Z; w) | |

| | | |
|---|---|---|
| 5: | M-Step: | $\begin{matrix}\begin{matrix}{π}_{m} , {Q}_{m} , {w}_{m} = \\ {arg max}_{π , Q , w} {𝔼}_{p ( Z , Z ( t ) \vert Y )} log P ( Y , Z , Z ( t ) ; π , Q , w )\end{matrix} & ( 16 )\end{matrix}$ |

The E-Step and M-Step in Algorithm 2 can be simplified for our construction of the patient disease trajectory model. The expectation of the first term of the RHS in Equation 15 is given by

$$\begin{matrix}{𝔼}_{P ( Z , Z ( t ) \vert Y ; {π}^{′} , {Q}^{′} , {w}^{′} )} log P ( Z , Z ( t ) ; π , Q ) = {\sum }_{Δ} {\sum }_{a , b ∈ K} {C}_{a b} ( Δ ) ( {\sum }_{c , d ∈ [ K ]} ( log {Q}_{cd} ) 𝔼 [ {𝒩}_{cd} ( Δ ) \vert Z ; {Q}^{′} ] - {Q}_{cd} 𝔼 [ {R}_{c} ( Δ ) \vert Z ; {Q}^{′} ] ) + \\ {𝔼}_{P ( Z , \vert Y ; {π}^{′} , {Q}^{′} , {w}^{′} )} log P ( {Z}_{{t}_{1}} ; π ) , \\ where & ( 17 ) \\ {C}_{a b} ( Δ ) {=}^{△} {\sum }_{n} {\sum }_{{t}_{2}}^{{t}_{n}} P ( {Z}_{n , {t}_{i - 1}} = a , {Z}_{n , {t}_{i}} = b \vert Y ; {π}^{′} , {Q}^{′} ) {1}_{{t}_{i} - {t}_{i - 1} = Δ} , & ( 18 )\end{matrix}$$

where [symbol]
(Δ) is the number of transitions between states a and b in time Δ and [symbol] ₐ (Δ) is the duration of time. The second term of the RHS in Equation 15 can be written as

$${E}_{P ( Z , \vert Y ; {π}^{′} , {Q}^{′} , {w}^{′} )} log P ( Y \vert Z ) = {\sum }_{n} {\sum }_{t = {t}_{1}}^{{t}_{n}} {\sum }_{k = 1}^{K} {\sum }_{d = 1}^{D} {γ}_{n , t , k} P ( {Y}_{n , t , d} \vert {Z}_{n . t} = k ) = {\sum }_{n} {\sum }_{t = {t}_{1}}^{{t}_{n}} {\sum }_{k = 1}^{K} {\sum }_{d = 1}^{D} {γ}_{n , t , k} {\sum }_{j} {w}_{k , d , j}^{{1}_{{Y}_{n , t , d} = {j}^{1} {Y}_{n , t , d}}} , \qquad ( 19 )$$

where γ_{n,t,k}=P(Z_{n,t}=k|Yₙ) is the posterior probability of disease state k for patient n at time point t, 1_{Y}_{_{n,t,d}} is an indicator of discrete bin.

The M-step in Equation 16 results in the following closed-form expressions for the parameters of the observation model and the initial probability vector:

$$\begin{matrix}{w}_{k , d , j} = \frac{{\sum }_{n} {\sum }_{t = {t}_{1}}^{{t}_{n}} {γ}_{n , t , k} {1}_{{Y}_{n , t , d}} {1}_{{Y}_{n , t , d} = j}}{{\sum }_{n} {\sum }_{t = {t}_{1}}^{{t}_{n}} {\sum }_{j} {γ}_{n , t , k} {1}_{{Y}_{n , t , d}} {1}_{{Y}_{n , t , d} = j}} & ( 20 ) \\ {π}_{a} = \frac{{\sum }_{n} P ( {Z}_{n , {t}_{1}} = a \vert {Y}_{n} ; {π}^{′} , {Q}^{′} )}{{\sum }_{n} {\sum }_{k = 1}^{K} P ( {Z}_{n , {t}_{1}} = k \vert {Y}_{n} ; {π}^{′} , {Q}^{′} )} & ( 21 )\end{matrix}$$

The generator matrix Q can be updated in each iteration using the closed-form solution:

$${Q}_{a b} = \frac{{\sum }_{Δ} {\sum }_{c , d ∈ [ K ]} 𝔼 [ \begin{matrix}{𝒩}_{a b} ( Δ ) \vert Z ( Δ ) = d , \\ Z ( 0 ) = c ; {Q}^{′}\end{matrix} ] {C}_{c , d} ( Δ )}{{\sum }_{Δ} {\sum }_{c , d ∈ [ K ]} 𝔼 [ \begin{matrix}{ℛ}_{a} ( Δ ) \vert Z ( Δ ) = d , \\ Z ( 0 ) = c ; {Q}^{′}\end{matrix} ] {C}_{c , d} ( Δ )} \qquad ( 22 )$$

The specific formulae for the involved terms are:

$$\begin{matrix} Q = U Λ {U}^{- 1} ( eigendecomposition ) & ( 23 ) \\ {χ}_{p q} ( Δ ) = \{ \begin{matrix}Δ exp ( Δ {Λ}_{p} ) & {Λ}_{p} = {Λ}_{q} \\ \frac{exp ( Δ {Λ}_{p} ) - exp ( Δ {Λ}_{q} )}{{Λ}_{p} - {Λ}_{q}} & {Λ}_{p} \neq {Λ}_{q}\end{matrix} & ( 24 ) \\ 𝔼 [ {ℛ}_{a} ( Δ ) \vert Z ( Δ ) = d , Z ( 0 ) = c ; {Q}^{′} ] = \frac{1}{{A}_{cd} ( Δ )} {\sum }_{p = 1}^{K} {U}_{c p} {U}_{p a}^{- 1} {\sum }_{q = 1}^{K} {U}_{a q} {U}_{q d}^{- 1} {χ}_{p q} ( Δ ) & ( 25 ) \\ 𝔼 [ {𝒩}_{ab} ( Δ ) \vert Z ( Δ ) = d , Z ( 0 ) = c ; {Q}^{′} ] = \frac{{Q}_{a b}}{{A}_{cd} ( Δ )} {\sum }_{p = 1}^{K} {U}_{c p} {U}_{p a}^{- 1} {\sum }_{q = 1}^{K} {U}_{bq} {U}_{q d}^{- 1} {χ}_{p q} ( Δ ) & ( 26 )\end{matrix}$$

Here,

A_{cd} (Δ) is the transition probability of moving from state c to d in time interval Δ (Equation 3). The ev [symbol] a, Z_{n,t}_{ᵢ}=b|Y; π′, Q′,w′) is done using the forward-backward algorithm for computing the posterior probabilities in hidden Markov models. Only the final results are given here. The approach consists of sequential updates of the form:

$$\begin{matrix} {c}_{n , {t}_{i}} α ( {Z}_{n , {t}_{i}} ) = P ( {Y}_{n , {t}_{i}} \vert {Z}_{n , {t}_{i}} ) {\sum }_{{Z}_{n , {t}_{i - 1}}} α ( {Z}_{n , {t}_{i - 1}} ) P ( {Z}_{n , {t}_{i}} \vert {Z}_{n , {t}_{i - 1}} ) and & ( 27 ) \\ {c}_{n , {t}_{i + 1}} β ( {Z}_{n , {t}_{i}} ) = {\sum }_{{Z}_{n , {t}_{i + 1}}} β ( {Z}_{n , {t}_{i + 1}} ) P ( {Y}_{n , {t}_{i + 1}} \vert {Z}_{n , {t}_{i + 1}} ) P ( {Z}_{n , {t}_{i + 1}} \vert {Z}_{n , {t}_{i}} ) , & ( 28 )\end{matrix}$$

where α(Z_{n,t}_{ᵢ})=P(Z_{n,t}_{ᵢ}|Yₙ) and β(Z_{n,t}_{ᵢ})=P(Y_{n,t}_{ᵢ₊₁}_{:t}_{ₙ}|Z_{n,t}_{ᵢ})/P(Y_{n,t}_{ᵢ₊₁}_{:t}_{ₙ}|Y_{n,t}_{₁}_{:t}_{ₙ}). Note c_{n,t} in the above equations is the coefficient that normalizes the RHS in Equation falphaUpdate. The marginal likelihood P(Y_{n,t}_{₁}_{:t}_{ₙ}), the posterior probability γ_{n,t,k}, and the bivariate marginal posterior probability P(Z_{n,t}_{_{i−1}}=a, Z_{n,t}_{ᵢ}=b|Yₙ; π′, Q′) can then be obtained as

$$\begin{matrix}P ( {Y}_{n} ) = {\prod }_{t = {t}_{1}}^{{t}_{n}} {c}_{n , t} , & ( 29 ) \\ {γ}_{n , {t}_{i} , k} = α ( {Z}_{n , {t}_{i}} ) β ( {Z}_{n , {t}_{i}} ) , and & ( 30 ) \\ P ( {Z}_{n , {t}_{i - 1}} , {Z}_{n , {t}_{i}} \vert {Y}_{n} ; {π}^{′} , {Q}^{′} , {w}^{′} ) = \frac{\begin{matrix}α ( {Z}_{n , {t}_{i - 1}} ) P ( {Y}_{n , {t}_{i}} \vert {Z}_{n , {t}_{i}} ) \\ P ( {Z}_{n , {t}_{i}} \vert {Z}_{n , {t}_{i - 1}} ) β ( {Z}_{n , {t}_{i}} )\end{matrix}}{{c}_{n , {t}_{i}}} . & ( 31 )\end{matrix}$$

When observed data is incomplete, that missing data is marginalized. As a result, incomplete data may be used to train and use the model.

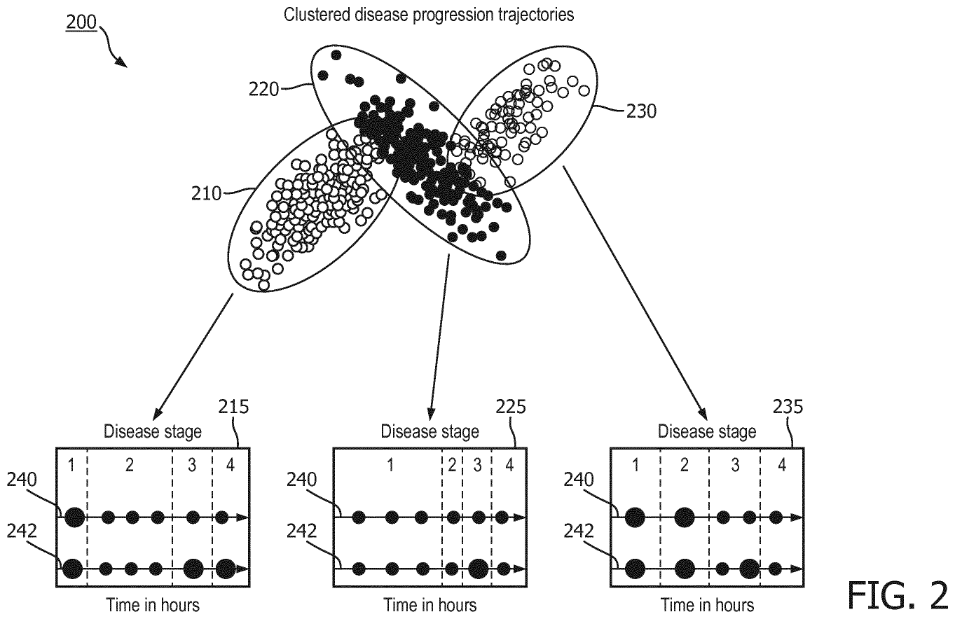

FIG. 2 illustrates prototypical results from the mixture-model model with three clusters. A cluster plot shows three different clusters 210, 220, and 230. For each cluster a disease state progression plot 215, 225, and 235 are also shown. In some cases, a specific patient may find themselves in different classes and the class with the highest likelihood may be chosen. The disease state progression plot shows how the disease progresses through the different disease states over time and the variation in two different markers 240 and 242. A large circle for the parameters 240 and 242 indicates larger values for the respective marker. For cluster 210, disease state 1 is short, then state 2 is much longer, with states 3 and 4 almost as short as state 1. For cluster 220, disease state 1 is long, then state 2 is much shorter, with states 3 and 4 almost as short as state 2. For cluster 230, disease states 1, 2, and 3 are about the same length, with state 4 slightly shorter than states 2, 3, and 4.

Disease states may be characterized by three different parameters: the temporal pattern of observations; probability that an observation is made at a specific time-point; and the magnitude of the observation itself. The probability of making an observation at a time-point refers to the likelihood that a clinical marker (e.g., heart-rate measurement or change in ventilator setting) is actually made. As a result, the clusters shown in FIG. 2 are determined by each of these three different parameters.

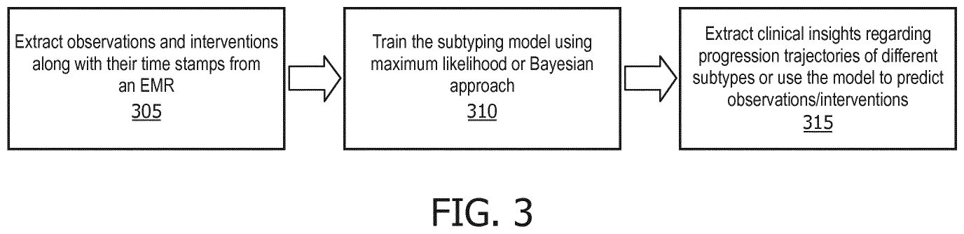

FIG. 3 is flow diagram illustrating the uses of the model by clinicians and hospitals to understand how disease development and management varies across different subtypes of any acute or chronic disease. First, observations and interventions along with their time stamps are extracted from patient EMRs 305. This extracted information is then used to train the subtyping model using the maximum likelihood or Bayesian approach 310 as described above. Next, clinical insights regarding the progression trajectories of different subtypes may be extracted 315. Also, the model may be used to predict the future course of observations and/or interventions for a new patient.

The use of this model has implications in understanding what observations/interventions to perform at what time of the patient's care continuum. The model may also be used to predict various outcomes such as how the patient's disease would evolve in time, how will a patient respond to certain interventions, what schedule of medications to provide for optimal recovery, how long will it take for the patient's condition to deteriorate, and other long-term outcomes like hospital length of stay, mortality etc. When a patient is admitted, a few measurements are taken along with the patient's history, and a treatment and care plan would be suggested by the model. Various suggested treatment plans may be tried to determine which one causes the most improvement in the patient. In addition to acting as a clinical decision support tool, the model may be used by researchers and drug designers to understand disease endotypes. At the patient level, the model may be used by patients as a personalized tool to monitor their progress and accurately predict when they are likely to need the attention of a clinician. Also, the models may help a hospital to learn about the different subtypes and learn what the optimal care plans are for patients in the different subtypes. For example, for ARDS patients with pneumonia, more continuous monitoring of their status is needed, say every hour certain measurements should be taken. In another example, a patient with ARDS based upon sepsis may be stable for 10 hours and then exhibit a steep decline. This would lead to a different monitoring and care plan. This allows for ICU resources to be appropriately assigned to different patients based upon their disease subtype.

The embodiments described herein may be utilized in hospitals and homes for management of acute and chronic diseases.

Now some examples of when disease subtyping from temporal progression modelling may be beneficial are presented.

Clinical practice has a lot of variability due to disease heterogeneity and clinician practice variabilities. Some of this variability is good as clinicians need to individualize care. Some of this variability may be undesirable when it deviates from best practice recommendations. Taking into account when measurements are made and their value, cluster disease trajectories may be clustered, and cases may be identified when clinical care may be outliers from dominant clusters. Attention may then be drawn to patients who may be under or over treated, and the clinicians may be asked to reevaluate the patient. This can take place during individual patient care as well as protocol reviews.

There is interest in predicting patient deterioration, and there are already a number of such algorithms to predict such patient deterioration: hemodynamic instability indicator, acute kidney injury, and acute respiratory distress syndrome. Existing scores only assess risk level without any timing prediction. By taking into account temporal patterns of measurements and having a latent representation of disease progression, a clinician will be able to determine when hemodynamic instability risk will cross a critical threshold. The output of the mode may state that: “Patient is likely to need cardiovascular interventions within 2 hrs. You may wish to evaluate the patient to determine if earlier intervention would be beneficial.”

Many conditions in critical care (sepsis, acute kidney injury, acute respiratory distress syndrome, etc.) and chronic conditions (heart failure, chronic kidney disease, etc.) are actually made up of subtypes with different underlying physiology and will progress differently. Taking into account the temporal patterns of clinical observations, these disease subtypes may be better identified. For example, patients with acute kidney injury who require dialysis and then recover were at especially high risk of progression to chronic kidney disease. In another example, studies have identified 3 subtypes in pediatric sepsis based on the time course of gene expression. The model described above will be able to identify subtypes of disease based on patient acuity, the interventions they receive.

Various features of the embodiments described above result in a technological improvement and advancement over existing disease modelling systems. Such features include, but are not limited to identifying disease subtypes with different disease progression profiles, producing a model to allow clinicians to better provide individualized care based upon patient specific data matching specific disease subtypes, and providing better predictions of patient disease progression. These models may be use with data that is always incomplete and irregularly sampled over varying time intervals without the need for an imputation step to produce a complete data set of constant dimensions.

The embodiments described herein may be implemented as software running on a processor with an associated memory and storage. The processor may be any hardware device capable of executing instructions stored in memory or storage or otherwise processing data. As such, the processor may include a microprocessor, field programmable gate array (FPGA), application-specific integrated circuit (ASIC), graphics processing units (GPU), specialized neural network processors, or other similar devices.

The memory may include various memories such as, for example L1, L2, or L3 cache or system memory. As such, the memory may include static random access memory (SRAM), dynamic RAM (DRAM), flash memory, read only memory (ROM), or other similar memory devices.

The storage may include one or more machine-readable storage media such as read-only memory (ROM), random-access memory (RAM), magnetic disk storage media, optical storage media, flash-memory devices, or similar storage media. In various embodiments, the storage may store instructions for execution by the processor or data upon with the processor may operate. This software may implement the various embodiments described above.

Further such embodiments may be implemented on multiprocessor computer systems, distributed computer systems, and cloud computing systems.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A method of determining patient subtyping from disease progression trajectories, comprising:
extracting patient data and related time stamps from patient record data related to a disease, wherein the extracted patient data comprises physiological data received via physiological sensors, and wherein the extracted patient data is incomplete and irregular;
building a continuous-time disease progression model based upon the extracted patient data;
building a mixture model for clustering of patient disease trajectory subtypes;
extracting clinical insights regarding disease progression from the patient disease trajectory subtypes; and
generating a care plan based on the extract clinical insights.

2. (canceled)

3. The method of claim 2, further comprising displaying clustered extracted patient data and a disease state diagram.

4. The method of claim 1, further comprising predicting a patient observation by inputting patient data into the mixture model to determine the patient's disease trajectory.

5. The method of claim 4, further comprising recommending a patient intervention based upon the predicted patient observation.

6. The method of claim 1, wherein the continuous-time disease progression model is a continuous Markov chain.

7. The method of claim 6, wherein the continuous-time disease progression model parameters are determined based upon training data.

8. The method of claim 1, wherein the mixture model is trained using a maximum likelihood approach.

9. The method of claim 1, wherein the mixture model is trained using a Bayesian approach.

10. A non-transitory machine-readable storage medium encoded with instructions for determining patient subtyping from disease progression trajectories, the non-transitory machine-readable storage medium comprising instructions for:
extracting patient data and related time stamps from patient record data related to a disease, wherein the extracted patient data comprises physiological data received via physiological sensors, and wherein the extracted patient data is incomplete and irregular;
building a continuous-time disease progression model based upon the extracted patient data;
building a mixture model for clustering of patient disease trajectory subtypes;
extracting clinical insights regarding disease progression from the patient disease trajectory subtypes; and
generating a care plan based on the extract clinical insights.

11. (canceled)

12. The non-transitory machine-readable storage medium of claim 11, further comprising displaying clustered extracted patient data and a disease state diagram.

13. The non-transitory machine-readable storage medium of claim 10, further comprising predicting a patient observation by inputting patient data into the mixture model to determine the patient's disease trajectory.

14. The non-transitory machine-readable storage medium of claim 13, further comprising recommending a patient intervention based upon the predicted patient observation.

15. The non-transitory machine-readable storage medium of claim 10, wherein the continuous-time disease progression model is a continuous Markov chain.

16. The non-transitory machine-readable storage medium of claim 15, wherein the continuous-time disease progression model parameters are determined based upon training data.

17. The non-transitory machine-readable storage medium of claim 10, wherein the mixture model is trained using a maximum likelihood approach.

18. The non-transitory machine-readable storage medium of claim 10, wherein the mixture model is trained using a Bayesian approach.
